(19) United States
(12) Patent Application Publication
(10) Pub. No.: US 2024/0394315 A1
(43) Pub. Date: Nov. 28, 2024

(54) LABORATORY PROCESS EXECUTION AND MONITORING SYSTEM

(71) Applicant: SAUDI ARABIAN OIL COMPANY, Dhahran (SA)

(72) Inventors: Hamoud A. Alqahtani, Dhahran (SA); Salah Hamad Al-Saleh, Dhahran (SA); Subhash Ayirala, Dhahran (SA); Ali Yousef, Dhahran (SA)

(73) Assignee: SAUDI ARABIAN OIL COMPANY, Dhahran (SA)

(21) Appl. No.: 18/324,882

(22) Filed: May 26, 2023

(51) Int. Cl. G06F 16/95

(52) U.S. Cl. CPC G06F 16/95; G01N 33/24; G01N 35/00871

(57) ABSTRACT

A system includes a core flood apparatus, a laboratory computing device configured to collect laboratory data when performing a core flooding test using the core flood apparatus, and a data control and data monitor server communicatively interfacing with the laboratory computing device and configured to receive the laboratory data, and send instructions to the laboratory computing device. The system further includes a data management and analysis facility that is physically separate from the laboratory computing device and the core flood apparatus, and configured to receive the laboratory data from the data control and data monitor server and store the laboratory data.

### BACKGROUND

Laboratory data collection is an important part of many processes, e.g., in the oil and gas industry. A laboratory instrument may gather data, e.g., from a sample to be analyzed. The data gathering may proceed over different time frames, e.g., seconds, minutes, hours, days, months, etc., and the gathered data may be received by a laboratory computer. The data gathering may be supervised by a technician or scientist, and eventually the data may be analyzed, and/or archived.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a system comprising: a core flood apparatus; a laboratory computing device configured to collect laboratory data when performing a core flooding test using the core flood apparatus; a data control and data monitor server communicatively interfacing with the laboratory computing device and configured to: receive the laboratory data, send instructions to the laboratory computing device; and a data management and analysis facility that is physically separate from the laboratory computing device and the core flood apparatus, and configured to: receive the laboratory data from the data control and data monitor server and store the laboratory data.

In general, in one aspect, embodiments relate to a system comprising: a laboratory instrument; a laboratory computing device configured to collect laboratory data from the laboratory instrument; a data control and data monitor server communicatively interfacing with the laboratory computing device and configured to: receive the laboratory data, send instructions to the laboratory instrument; a data management and analysis facility that is physically separate from the laboratory computing device and the laboratory instrument, and configured to: receive the laboratory data from the data control and data monitor server and store the laboratory data; and an enforcement zone using a Pattern 3+Absolute Enforcement approach.

In general, in one aspect, embodiments relate to a method comprising: receiving, by a data control and monitor server, laboratory data from a laboratory computing device, wherein the laboratory data originate from a core flooding test performed using a core flood apparatus; providing, by the data control and monitor server, the laboratory data to a data management and analysis facility.

In light of the structure and functions described above, embodiments of the disclosure may include respective means adapted to carry out various steps and functions defined above in accordance with one or more aspects and any one of the embodiments of one or more aspect described herein.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows a laboratory process execution and monitoring system in accordance with one or more embodiments.

FIG. 2 shows a laboratory process execution and monitoring system in accordance with one or more embodiments.

FIG. 3 shows a laboratory process execution and monitoring system in accordance with one or more embodiments.

FIG. 4 shows a laboratory process execution and monitoring system in accordance with one or more embodiments.

FIG. 5 shows a flowchart for a method in accordance with one or more embodiments.

FIG. 6 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

A laboratory process may involve many operations such as collecting data using a lab instrument, processing the data, analyzing the data, securing the data, sharing the data. In addition, the laboratory process may require significant monitoring (e.g., of an ongoing collection of data from a sample) and may involve commands to control or adjust the laboratory process. In a traditional configuration, data acquisition is performed by a lab instrument that is coupled to a laboratory computer. In this configuration, data may be locally collected and stored on the laboratory computer. A user of the laboratory computer may monitor and control the data collection. In this traditional configuration data management and data analysis may be performed elsewhere, with no functional link to the laboratory computer. In other words, the traditional configuration is sectored, with a first sector for the data collection and a second separate sector for data management and data analysis.

In general, embodiments of the disclosure include systems and methods for executing and monitoring a laboratory process. In the embodiments as subsequently described, the sectoring of the traditional configuration is reduced or eliminated. The proposed system includes a data flow architecture and may be based on an edge-fog-cloud architecture and network to automate the laboratory data gathering process and/or to provide remote access to the laboratory facilities. Laboratory productivity, laboratory and personal safety, data integrity and data management, and remote process management are a few of the advantages of the described embodiments. These and other benefits may be realized by the strategic addition of a server between laboratory-based equipment and the facility for data management, without fundamentally altering certain components of the traditional configuration. A detailed description is subsequently provided in reference to the figures.

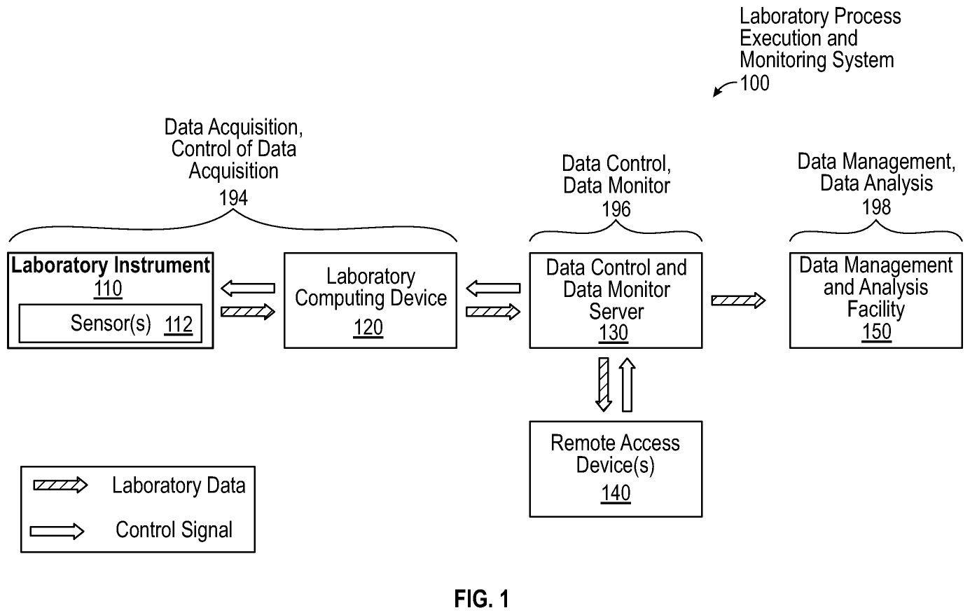

FIG. 1 shows a laboratory process execution and monitoring system (100) in accordance with one or more embodiments. The system includes components for data acquisition and the control of data acquisition (194), components for data control and data monitoring (196) and components for data management and data analysis (198). Laboratory data and control signals are exchanged between these components as subsequently discussed.

The components for data acquisition and the control of data acquisition (194), in one or more embodiments, include a laboratory instrument (110) and a laboratory computing device (120).

The laboratory instrument (110) may be any type of laboratory instrument that produces one or more laboratory data, e.g., based on one or more measurements being performed using the laboratory instrument (110). In one embodiment, the laboratory instrument is configured to perform a core flooding laboratory test that introduces fluid, such as water, into a rock sample to measure interactions between the fluid and the rock. The rock sample used in core flooding may come from a reservoir, for example, to measure feasibility of a waterflooding process. In the field of oil and gas, waterflooding is a process to increase production from hydrocarbon-bearing reservoirs. A waterflooding process injects water into a hydrocarbon-producing reservoir. The hydrocarbons within the reservoir are displaced by the water and is pushed toward an adjacent production well. The displaced hydrocarbons are collected and produced.

Core flooding is widely used in the petroleum industry. Conventionally performed, it is time consuming, requiring several weeks to months to be completed. Accordingly, automation of core flooding, including data collection, with the option to perform and monitor it remotely, is highly desirable. The ability to remotely perform these operations, in accordance with embodiments of the disclosure, improves data integrity, and enables continuous execution of the core flooding, without interruptions. Further, with core flooding involving high temperatures and/or pressures, remote monitoring results in improved safety. Emergency situations can be quickly identified and reported.

The laboratory instrument (110) used for a core flooding test is a core flood apparatus and may be found in in a petroleum research lab. The core flood apparatus may be used to measure permeability, relative permeability, saturation change, formation damage caused by the fluid injection, and/or interactions between the fluid and the rock. The core material may come from an oil reservoir, but tests may also use outcrop rock. The fluid in place at the start of the test may be a simulated formation brine, oil (either crude oil or refined oil), or a combination of brine and oil. Injected fluids may include crude oil, simulated reservoir brine, refined fluids, drilling mud filtrate, acids, foam or other chemicals used in the oil field. Depending on the purpose of the test, conditions may be either ambient temperature and low confining pressure or high temperature and pressure of a subject reservoir. Pressures and flow rates at both ends of the core are measured, and the core may further be investigated using other measurements such as nuclear magnetic resonance (NMR) during the test. Core flooding is typically used to determine the optimum development option for an oil reservoir and often helps evaluate the effect of injecting fluids specially designed to improve or enhance oil recovery.

In the example of a core flood apparatus, variables such a pressures, flow rates, temperatures, etc. may be measured by sensors (112) and included in the laboratory data.

Other data may be measured by other types of laboratory instruments. The laboratory instrument (110) may be any type of laboratory instrument equipped with any type and number of sensors (112).

The laboratory computing device (120) may be communicatively interfaced with the laboratory instrument (110) in order to receive the laboratory data. The laboratory data may be received in any form, e.g., in the form of analog voltages or currents, digital signals, optical signals, etc. The laboratory computing device (120) may be equipped with appropriate interfaces to receive the laboratory data. For example, the laboratory computing device (120) may be equipped with analog-to-digital converters. The laboratory computing device (120) may be equipped with storage to store, temporarily or permanently, the laboratory data. The laboratory computing device (120) may be equipped with a user interface to visualize the laboratory data for a local user of the laboratory computing device (120).

In one or more embodiments, the laboratory computing device (120) is configured to send a control signal to the laboratory instrument (110) in order to control the operation of the laboratory instrument (110). For example, the laboratory computing device (120) may send a control signal to control a pump to regulate the pressure of a fluid of the core flood apparatus. In the configuration as described, a local user of the laboratory computing device (120) may thus locally control the acquisition of measurements in the form of laboratory data by the laboratory instrument (110).

In one or more embodiments, the laboratory computing device (120) is equipped with a network interface (e.g., an Ethernet network interface) to communicate with other equipment such as the data control and data monitor server (130), described below.

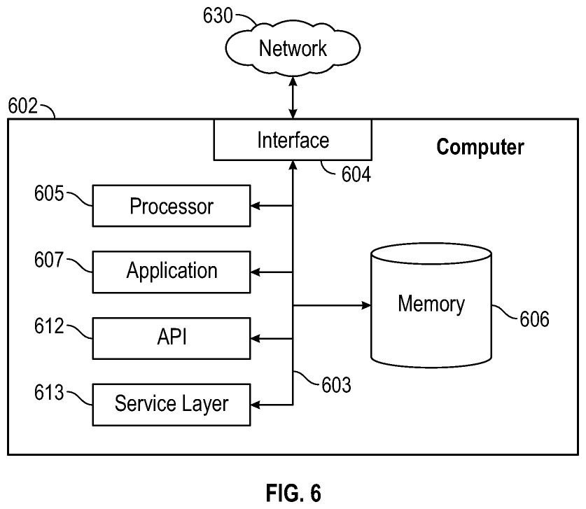

The laboratory computer device maybe a computing system as described in reference to FIG. 6. The computer system may be an integral part of the laboratory instrument (110), or it may be physically separate.

The components for data control and data monitoring (196), in one or more embodiments, include a data control and data monitor server (130). The data control and data monitor server (130) may be a computing system as described in reference to FIG. 6 and may be equipped with storage media for temporarily or permanently storing data obtained from the laboratory instrument (110). A network interface communicatively interfaces the data control and data monitor server (130) with the laboratory computing device (120). The data control and data monitor server (130) may further allow additional remote access devices (140) to connect.

The remote access devices (140) may enable remote users to access the laboratory computing device (120) via the data control and data monitor server (130) which may forward control signals to the laboratory computing device (120). The remote access devices (140) may receive laboratory data and may also issue control signals. Accordingly, users of the remote access devices (140) may remotely view data obtained from the laboratory instrument (110) and may further remotely control the operation of the laboratory instrument (110).

In one or more embodiments, the data control and data monitor server (130) further provides an interface to a data management and analysis facility (150), described below. The control and data monitor server (130) may send requests for data to the laboratory computing device (120) and may collect and send data (either live data from the laboratory instrument (110) or archived data) once requested by the data management and analysis facility (150).

The components for data management and data analysis (198), in one or more embodiments, include a data management and analysis facility (150). The data management and analysis facility (150) may include a data storage. The data management and analysis facility (150) may be able to read data coming in from the data control and data monitor server (130), but it may not be able to send requests for data. The data storage in the data management and analysis facility (150) may be a duplicate of a similar data storage in the data control and data monitor server (130).

While FIG. 1 shows various configurations of hardware components and/or software components, other configurations may be used without departing from the scope of the disclosure. For example, while a single laboratory instrument (110) is shown, the system (100) may support any number of laboratory instruments of any type. Embodiments of the disclosure may be particularly suitable for laboratory analyses that are performed over longer times and that involve harsh experimental conditions. Also, while not explicitly shown in FIG. 1, the components may be at particular locations. For example, the components associated with data acquisition and control of data acquisition (194) may be part of a lab facility. The components associated with data control and data monitor (196) may be located elsewhere, either locally or remote, and the data management and data analysis (198) may be in an IT building, and administrative building, etc. Other locations may be chosen without departing from the disclosure. Further, various components in FIG. 1 may be combined to create a single component. As another example, the functionality performed by a single component may be performed by two or more components. While one is shown, any number.

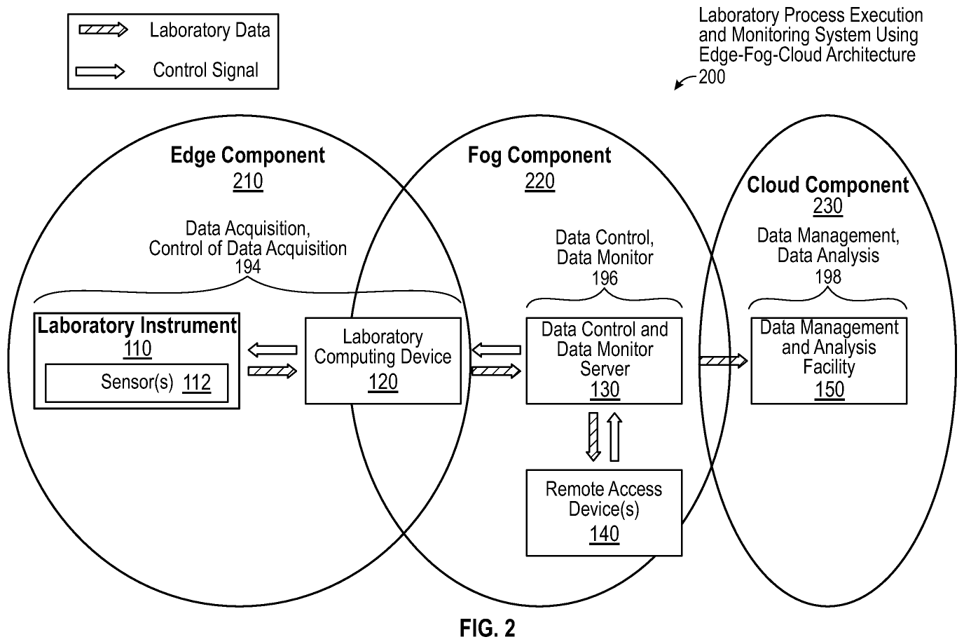

FIG. 2 shows a laboratory process execution and monitoring system (200) using an edge-fog-cloud architecture, e.g., as used in Industry 4.0 environments, in accordance with one or more embodiments. Conceptually, the components such as the laboratory instrument (110), the laboratory computing device (120), the data control and data monitor server (130), the remote access device(s) (140), and the data management and analysis facility (150) may be as previously described in reference to FIG. 1. However, FIG. 2 shows the organization of these components in an edge-fog-cloud architecture.

Edge computing may be defined as the processing of laboratory data away from a centralized nodes (such as the data management and analysis facility (150)) and close to the logical edge of the network, toward individual sources of data (such as the laboratory instrument (110)). It effectively pushes the computational functions to the edge component (210) of the edge-fog-cloud architecture. In other words, rather than requiring the transmission of all laboratory data to the cloud for analysis and action, this process may take place much closer to the data source.

Edge computing may triage the laboratory data locally, thereby reducing the backhaul traffic to a central repository at the data control and data monitor server (130) and/or the data management and analysis facility (150). This may simplify or reduce the communications via the fog component (220) and may reduce potential points of failure.

Fog computing may be defined as an extension of a cloud component (230) of a network towards the edge component (210) of the network. Using a fog component (220), computational resources are made available on a local area network (LAN) level of the network. Laboratory data that would otherwise be processed by the data management and analysis facility (150) in the cloud component (230) of the network may be processed locally at the level of the data control and data monitor server (130).

Cloud computing may be used to store, process, and/or accesses the laboratory data over the internet. The cloud component (230) may include the data management and analysis facility (150) to provide storage and processing resources.

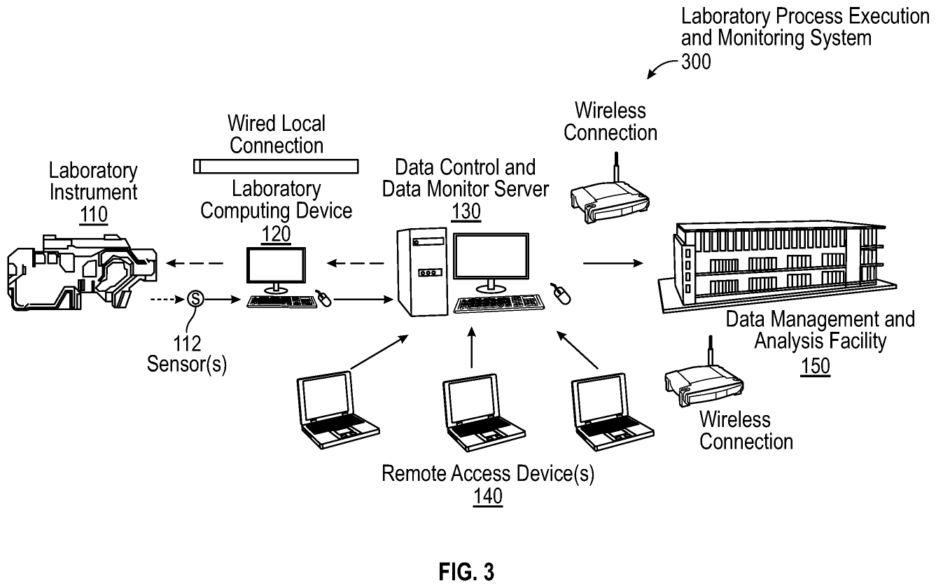

FIG. 3 shows a laboratory process execution and monitoring system (300) in accordance with one or more embodiments. FIG. 3 provides additional details on the components introduced in reference to FIGS. 1 and 2. Specifically, FIG. 3 shows the components that may be used to implement the previously described configurations and further identifies possible wired and wireless network segments that may be used to interface the components. In the example, the data control and data monitor server (130) interfaces with the laboratory computing device (120) through a wired connection. Wireless connections may be used to interface the remote access devices (140) with the data control and data monitor server (130).

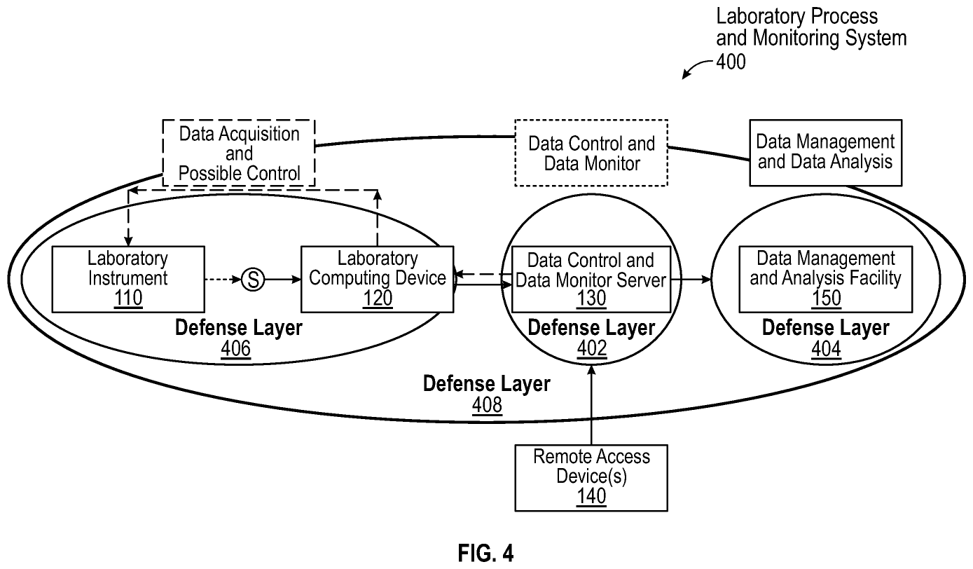

FIG. 4 shows a laboratory process execution and monitoring system (400) in accordance with one or more embodiments. A configuration based on a defense in layer model is shown. The system (400) includes multiple defense layers (402, 404, 406, 408). In comparison to conventional implementations, the system (400) includes an extra layer of defense (402) implemented around the data control and data monitor server (130) to provide protection on all interfaces, as further discussed below. In addition, the system (400) includes a layer of defense (404) implemented around the data management and analysis facility (150), a layer of defense (406) around the laboratory computing device (120) and the laboratory instrument (110), and a layer of defense (408) around the entire network.

In one or more embodiments of the disclosure, components of the system (400) are implemented using a PI system by OSIsoft, LLC. The data control and data monitor server (130) may implement an enforcement zone using a Pattern 3+ Absolute Enforcement approach. In the Pattern 3+ Absolute Enforcement approach, a physically one-way data link (e.g., fiber-optically implemented) is used to limit certain traffic from the laboratory computing device (120) to elsewhere (e.g., the data management and analysis facility (150), while blocking traffic in the opposite direction. The approach enables signals (e.g., control signals) to be sent from the data control and data monitor server (130) and from the laboratory computing device (120) to the laboratory instrument (110). However, embodiments of the disclosure ensure that no control signal can be sent from the data management and analysis facility (150) or from other external locations aside from the remote access devices (140).

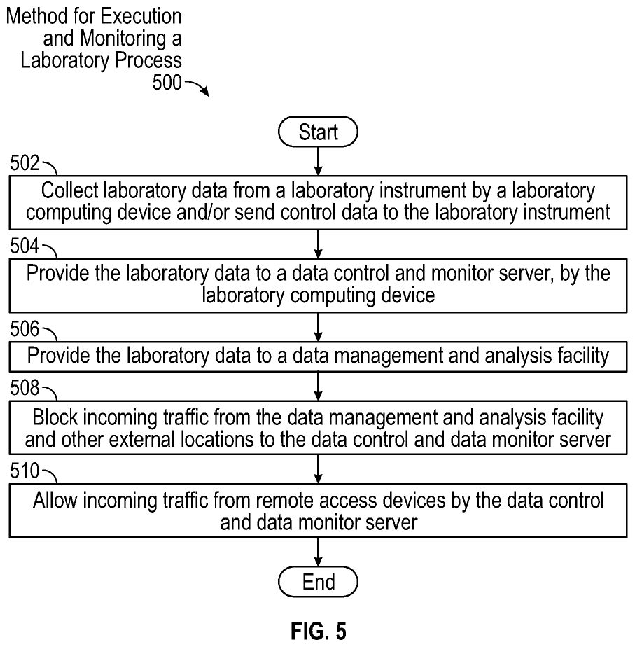

FIG. 5 shows a flowchart in accordance with one or more embodiments. One or more steps in FIG. 5 may be performed by one or more components shown in FIGS. 1-4. While the various steps in FIG. 5 are presented and described sequentially, one of ordinary skill in the art will appreciate that some or all of the steps may be executed in different orders, may be combined or omitted, and some or all of the steps may be executed in parallel. Furthermore, the blocks may be performed actively or passively.

In Step 502, laboratory data are obtained by a laboratory computing device. The laboratory data may originate from a laboratory instrument, for example, a core flood apparatus performing a core flooding test.

In Step 504, the laboratory data are provided to a data control and monitor server, by the laboratory computing device.

In Step 506, the laboratory data are provided to a data management and analysis facility.

In Step 508, incoming traffic from the data management and analysis facility and other external locations to the data control and data management server is blocked.

In Step 510, an exception allows incoming traffic from remote access devices to reach the data control and data monitor server.

Embodiments of the disclosure have at least one or more of the following advantages. Embodiments of the disclosure provide systems and methods to automate the laboratory data gathering process and provide remote access in the laboratory facilities. By introducing a data control and data monitoring server, laboratory test results may be accessible from different locations, and laboratory tests may be requested and controlled from different locations, thereby increasing laboratory productivity.

Embodiments of the disclosure further allow laboratory technicians to monitor test remotely and report for emergency situations as soon they occur. This may be particularly beneficial during situations such as COVID-19. Laboratory and personal safety are thus enhanced.

Further, data integrity and data management are improved by introducing the data control and data monitor server, which allows data to be stored on either side of the server. Data may thus be redundant, which is beneficial in case of a system failure.

Remote Processes Management may benefit from the data control and data monitor server being accessible from different devices. Security is maintained through identification and authentication protocols, remote-control protocols, cyber authorization protocols, and data transfer protocols.

The architecture supports future enhancements by connecting any system to the data control and data monitor server.

Also, the introduction of the data control and data monitor server is straightforward because no major alteration of an existing system is needed.

Embodiments may be implemented on a computer system. FIG. 6 is a block diagram of a computer system (602) used to provide computational functionalities associated with described algorithms, methods, functions, processes, flows, and procedures as described in the instant disclosure, according to an implementation. The illustrated computer (602) is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (602) may include a computer that includes an input device, such as a keypad, keyboard, touch screen, or other device that can accept user information, and an output device that conveys information associated with the operation of the computer (602), including digital data, visual, or audio information (or a combination of information), or a GUI.

The computer (602) can serve in a role as a client, network component, a server, a database or other persistency, or any other component (or a combination of roles) of a computer system for performing the subject matter described in the instant disclosure. The illustrated computer (602) is communicably coupled with a network (630). In some implementations, one or more components of the computer (602) may be configured to operate within environments, including cloud-based, local, global, or other environment (or a combination of environments).

At a high level, the computer (602) is an electronic computing device operable to receive, transmit, process, store, or manage data and information associated with the described subject matter. According to some implementations, the computer (602) may also include or be communicably coupled with an application server, e-mail server, web server, caching server, streaming data server, business intelligence (BI) server, or other server (or a combination of servers).

The computer (602) can receive requests over network (630) from a client application (for example, executing on another computer (602)) and responding to the received requests by processing the said requests in an appropriate software application. In addition, requests may also be sent to the computer (602) from internal users (for example, from a command console or by other appropriate access method), external or third-parties, other automated applications, as well as any other appropriate entities, individuals, systems, or computers.

Each of the components of the computer (602) can communicate using a system bus (603). In some implementations, any or all of the components of the computer (602), both hardware or software (or a combination of hardware and software), may interface with each other or the interface (604) (or a combination of both) over the system bus (603) using an application programming interface (API) (612) or a service layer (613) (or a combination of the API (612) and service layer (613). The API (612) may include specifications for routines, data structures, and object classes. The API (612) may be either computer-language independent or dependent and refer to a complete interface, a single function, or even a set of APIs. The service layer (613) provides software services to the computer (602) or other components (whether or not illustrated) that are communicably coupled to the computer (602). The functionality of the computer (602) may be accessible for all service consumers using this service layer. Software services, such as those provided by the service layer (613), provide reusable, defined business functionalities through a defined interface. For example, the interface may be software written in JAVA, C++, or other suitable language providing data in extensible markup language (XML) format or other suitable format. While illustrated as an integrated component of the computer (602), alternative implementations may illustrate the API (612) or the service layer (613) as stand-alone components in relation to other components of the computer (602) or other components (whether or not illustrated) that are communicably coupled to the computer (602). Moreover, any or all parts of the API (612) or the service layer (613) may be implemented as child or sub-modules of another software module, enterprise application, or hardware module without departing from the scope of this disclosure.

The computer (602) includes an interface (604). Although illustrated as a single interface (604) in FIG. 6, two or more interfaces (604) may be used according to particular needs, desires, or particular implementations of the computer (602). The interface (604) is used by the computer (602) for communicating with other systems in a distributed environment that are connected to the network (630). Generally, the interface (604) includes logic encoded in software or hardware (or a combination of software and hardware) and operable to communicate with the network (630). More specifically, the interface (604) may include software supporting one or more communication protocols associated with communications such that the network (630) or interface's hardware is operable to communicate physical signals within and outside of the illustrated computer (602).

The computer (602) includes at least one computer processor (605). Although illustrated as a single computer processor (605) in FIG. 6, two or more processors may be used according to particular needs, desires, or particular implementations of the computer (602). Generally, the computer processor (605) executes instructions and manipulates data to perform the operations of the computer (602) and any algorithms, methods, functions, processes, flows, and procedures as described in the instant disclosure.

The computer (602) also includes a memory (606) that holds data for the computer (602) or other components (or a combination of both) that can be connected to the network (630). For example, memory (606) can be a database storing data consistent with this disclosure. Although illustrated as a single memory (606) in FIG. 6, two or more memories may be used according to particular needs, desires, or particular implementations of the computer (602) and the described functionality. While memory (606) is illustrated as an integral component of the computer (602), in alternative implementations, memory (606) can be external to the computer (602).

The application (607) is an algorithmic software engine providing functionality according to particular needs, desires, or particular implementations of the computer (602), particularly with respect to functionality described in this disclosure. For example, application (607) can serve as one or more components, modules, applications, etc. Further, although illustrated as a single application (607), the application (607) may be implemented as multiple applications (607) on the computer (602). In addition, although illustrated as integral to the computer (602), in alternative implementations, the application (607) can be external to the computer (602).

There may be any number of computers (602) associated with, or external to, a computer system containing computer (602), each computer (602) communicating over network (630). Further, the term “client,” “user,” and other appropriate terminology may be used interchangeably as appropriate without departing from the scope of this disclosure. Moreover, this disclosure contemplates that many users may use one computer (602), or that one user may use multiple computers (602).

In some embodiments, the computer (602) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, a cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AlaaS), and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A system, comprising:
a core flood apparatus;
a laboratory computing device configured to collect laboratory data when performing a core flooding test using the core flood apparatus;
a data control and data monitor server communicatively interfacing with the laboratory computing device and configured to:
receive the laboratory data,
send instructions to the laboratory computing device; and
a data management and analysis facility that is physically separate from the laboratory computing device and the core flood apparatus, and configured to:
receive the laboratory data from the data control and data monitor server and store the laboratory data.

2. The system of claim 1, further comprising:
a remote access device, configured to receive the laboratory data via the data control and data monitor server.

3. The system of claim 2, wherein the remote access device is further configured to send control signals to the laboratory computing device via the data control and data monitor server.

4. The system of claim 1, wherein the data control and data monitor server is further configured to block incoming traffic with an exception allowing incoming traffic from remote access devices.

5. The system of claim 1, further comprising an enforcement zone using a Pattern 3+ Absolute Enforcement approach.

6. The system of claim 5, wherein the enforcement zone comprises a physically one-way data link between the data control and data monitor server and the data management and analysis facility.

7. The system of claim 1, wherein the data management and analysis facility is cloud-based.

8. The system of claim 1, organized in an edge-fog-cloud architecture, wherein:
an edge component of the edge-fog-cloud architecture comprises the laboratory computing device,
a fog component of the edge-fog-cloud architecture comprises the data control and data monitor server, and
a cloud component of the edge-fog-cloud architecture comprises the data management and analysis facility.

9. A system, comprising:
a laboratory instrument;
a laboratory computing device configured to collect laboratory data from the laboratory instrument;
a data control and data monitor server communicatively interfacing with the laboratory computing device and configured to:
receive the laboratory data, and
send instructions to the laboratory instrument;
a data management and analysis facility that is physically separate from the laboratory computing device and the laboratory instrument, and configured to:
receive the laboratory data from the data control and data monitor server and store the laboratory data; and
an enforcement zone using a Pattern 3+ Absolute Enforcement approach.

10. The system of claim 9, further comprising:
a remote access device, configured to receive the laboratory data via the data control and data monitor server.

11. The system of claim 10, wherein the remote access device is further configured to send control signals to the laboratory computing device via the data control and data monitor server.

12. The system of claim 9, wherein the data control and data monitor server is further configured to block incoming traffic with an exception allowing incoming traffic from remote access devices.

13. The system of claim 9, wherein the enforcement zone comprises a physically one-way data link between the data control and data monitor server and the data management and analysis facility.

14. The system of claim 9, wherein the data management and analysis facility is cloud-based.

15. The system of claim 9, organized in an edge-fog-cloud architecture, wherein:
an edge component of the edge-fog-cloud architecture comprises the laboratory computing device,
a fog component of the edge-fog-cloud architecture comprises the data control and data monitor server, and
a cloud component of the edge-fog-cloud architecture comprises the data management and analysis facility.

16. A method comprising:
receiving, by a data control and monitor server, laboratory data from a laboratory computing device, wherein the laboratory data originate from a core flooding test performed using a core flood apparatus;
providing, by the data control and monitor server, the laboratory data to a data management and analysis facility.

17. The method of claim 16, further comprising:
blocking incoming traffic from the data management and analysis facility.

18. The method of claim 17, wherein the blocking of incoming traffic is implemented using a Pattern 3+ Absolute Enforcement approach.

19. The method of claim 16, further comprising:
allowing incoming traffic from remote access devices.

20. The method of claim 19,
wherein the incoming traffic comprises control signals for controlling the core flood apparatus, and
wherein the laboratory computing device forwards the control signals to the laboratory computing device.
